(19) United States
(12) Patent Application Publication
(10) Pub. No.: US 2010/0209470 A1
(43) Pub. Date: Aug. 19, 2010

(54) DEMINERALIZED BONE MATRIX DEVICES

(75) Inventors: Suneeth Elizabeth Mohan, Memphis, TN (US); Susan J. Drapeau, Cordova, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC. An Indiana Corporation, Warsaw, IN (US)

(21) Appl. No.: 12/695,925

(22) Filed: Jan. 28, 2010

Related U.S. Application Data
(63) Continuation-in-part of application No. 11/415,036, filed on May 1, 2006.

(51) Int. Cl. A61K 35/32; A61K 9/10; A61F 2/00; A61P 19/08

(52) U.S. Cl. 424/423; 424/549; 424/443

(57) ABSTRACT

The invention relates to a sheet-form demineralized bone matrix (DBM) composition comprising an aqueous admixture of DBM particles, starch and gelatin, wherein the DBM particles are present in an amount of between about 30% and about 70% by weight, the gelatin is present in an amount between about 10 and about 40% by weight and the starch is present in an amount between about 5% and about 20% by weight, wherein the DBM particles are retained in the form of the resilient sheet.

### CROSS-REFERENCE TO RELATED APPLICATIONS

This application is a continuation-in-part of U.S. application Ser. No. 11/415,036, filed May 1, 2006, the entirety of which is incorporated by reference.

### BACKGROUND

The present invention relates generally to medical implants, more particularly, the present invention relates to osteoinductive medical products containing demineralized bone matrix.

A wide variety of implant formulations have been suggested in the art for the treatment of bone defects. In addition to traditional bone grafting, a number of synthetic bone graft substitutes have been used or explored, including some materials that contain demineralized bone matrix. Demineralized bone matrix has been shown to exhibit the ability to induce and/or conduct the formation of bone. It is thus desirable to implant and maintain demineralized bone matrix at a site at which bone growth is desired.

However, the beneficial nature of demineralized bone matrix is susceptible to disruption by the incorporation of incompatible materials or techniques when formulating the medical implant. At the same time, it is desirable to have implant products exhibiting good physical integrity to retain the demineralized bone matrix at the implant site, and that handle well in the operating environment and during implantation. As well, it is of considerable commercial significance that the formulation be manufacturable without undue cost, equipment or material burdens.

In view of the background in the area of demineralized bone matrix products, there exist a need for a product configuration that exhibits the ability to induce and/or support bone growth through the desired region and that has acceptable handling properties for surgeons.

Any publications or references discussed herein are presented to describe the background of the invention and to provide additional detail regarding its practice. Nothing herein is to be construed as an admission that the inventors are not entitled to antedate such disclosure by virtue of prior invention. Should there be a conflict, or apparent conflict, between the specification and any of the incorporated references, the specification takes precedence and the conflicting or apparently conflicting aspect of the reference is to be disregarded.

### SUMMARY OF THE INVENTION

In an exemplary embodiment, the present invention provides a sheet-form demineralized bone matrix (DBM) product providing a bone growth scaffold for use in medical situations where bone regeneration is desired. The product comprises a resilient sheet including DBM material and gelatin, which may optionally further include a starch and cosolvent, such as glycerol. The DBM particles are bound together using the gelatin, and optionally the gelatin and starch, without chemical cross-linking. Optionally, the final form, or shaped device, may be dehydrated. The DBM material retained in the resilient sheet exhibits osteoinductivity. In another exemplary embodiment, the sheet-form DBM product comprises DBM particles, gelatin, a starch and a cosolvent, such as glycerol. In another exemplary embodiment, the sheet-form DBM product consists essentially of DBM particles, gelatin, a starch and a cosolvent, such as glycerol.

In another exemplary embodiment, the invention provides a pre-formed flexible bone graft extender or bone void filler. The product includes a three-dimensionally stable, yet flexible, porous implant structure which comprises gelatin, DBM, starch and glycerol.

In another exemplary embodiment, the invention provides a pre-formed flexible bone graft extender or bone void filler possessing a certain amount of compression resistance in order to maintain space in a bone defect or cavity and allow regrowth of bone in the defect site or cavity. The compression resistance may be sufficient to minimize deformation by the surrounding musculature.

In another exemplary embodiment, the invention provides a method for preparing a demineralized bone matrix (DBM) implant material. The method includes preparing an admixture including gelatin, starch, glycerol and DBM. An admixture of gelatin, starch and glycerol in a liquid medium are heated and mixed to form a homogeneous mixture, the homogeneous mixture is then cooled prior to the addition of the DBM. In addition, the method may include the steps of mixing the cooled homogeneous mixture with DBM particles to form a DBM plus binder mixture at or below about 40° C., 39° C., 38° C., 37° C., 36° C. or 35° C. that may then be pored into a mold, thereby yielding a pre-formed flexible sheet material that may be used as a bone graft extender or bone void filler. In another exemplary embodiment, the pre-formed flexible sheet material may be dried or partially dried. For example, the pre-formed flexible sheet material may be lyophilized or partially lyophilized. In another exemplary embodiment, the outer perimeter of the pre-formed flexible sheet material is dried so as to create a pre-formed flexible sheet having a dried outer perimeter area and a substantially hydrated core area.

In another exemplary embodiment, the invention provides a method for preparing a demineralized bone matrix (DBM) implant material. The method includes providing an admixture including a gelatin, starch, glycerol and DBM in a liquid medium, with the gelatin and DBM present in a weight ratio of about 1:1 to about 1:4. The composition may also include a starch and glycerol in a weight ratio of about 1:1 or about 1:0.9 and/or the glycerol and DBM may be present in a weight ratio of about 1:2 to about 1:5 or about 1:3 to about 1:4. A method of preparing the devices may optionally include the further steps of drying or semi-drying the material. In an exemplary embodiment, the material placed in a mold will have a water content of from about 30% to about 70%, about 35% to about 70%, 40% to about 70%, 45% to about 70%, 30% to about 65%, 30% to about 60%, 30% to about 55%, 35% to about 65%, or 35% to about 60%.

In yet another exemplary embodiment, the gelatin has a gel strength between about 50 to about 300 or from about 200 to about 300 g Bloom.

In another exemplary embodiment, the invention provides a method for preparing a demineralized bone matrix (DBM) implant material. The method includes providing an admixture including a gelation compound (such as gelatin), a starch, glycerol and DBM in a liquid medium, with the gelation compound present in an amount between about 17% to about 30%, between about 15% to about 32%, between about 13% to about 34%, or about 10% to about 40% (based on dry weight), the DBM is present in an amount between about 41% to about 56%, between about 40% to about 60%, between about 35% to about 65% or about 30% to about 70% (based on dry weight), the starch may be present in an amount between about 12% to about 13%, between about 11% to about 14%, between about 10% to about 15% or between about 5% to about 20% (based on dry weight), glycerol is present in an amount between about 15 wt % to about 17%, between about 14% to about 18%, between about 13% to about 19% or between about 10% to about 20% (based on calculated weight) and the water content in the finished product is from about 30% to about 70%, about 35% to about 70%, 40% to about 70%, 45% to about 70%, 30% to about 65%, 30% to about 60%, 30% to about 55%, 35% to about 65%, or 35% to about 60%. The composition may also be dried or semi-dried.

In another exemplary embodiment, the invention provides a method for preparing a demineralized bone matrix (DBM) implant material. The method includes providing an admixture including DBM particles in a liquid medium containing a starch, gelatin and glycerol. The liquid medium is contacted with the DBM particles at a reduced temperature so as to preserve the function of the DBM particles. The method may optionally include the additional steps of freeze drying the product.

In still a further exemplary embodiment, the invention provides methods of treating patients comprising implanting in the patients DBM products as described herein, and medical products including such products packaged in sterile condition.

In still a further exemplary embodiment, the invention provides a DBM product substantially free of cortical cancellous bone chips. In yet another exemplary embodiment, the invention provides a DBM product that may be mixed with autograft, blood, blood derivatives, such as platelets and/or stem cells, bone marrow, bone marrow extract and bone marrow derivatives, such as platelets and/or stem cells. In yet another exemplary embodiment, the invention provides a DBM product that contains less than about 5%, about 4%, about 3%, about 2%, about 1%, about 0.5%, about 0.3% or about 0.1% residual calcium.

In another exemplary embodiment, the invention may further include a natural or synthetic calcium phosphate material, such as a tricalcium phosphate (α and/or β), hydroxyapatite, Bioglass®, biphasic calcium phosphate, corraline hydroxyapatite, and biocompatible ceramics. Biphasic calcium phosphate is a particular synthetic ceramic that may be used in the invention, for example, a tricalcium phosphate:hydroxyapatite mixture with a weight ratio of about 50:50 to about 95:5, about 70:30 to about 95:5, about 80:20 to about 90:10, or about 85:15. The calcium phosphate material may be in a granular form having an average particle diameter between about 0.2 and 5.0 mm, between about 0.4 and 3.0 mm, between about 0.4 and 2.0 mm or between about 0.5 and 1.0 mm.

In yet another exemplary embodiment, the invention provides an implant fabricated from a DBM material and gelatin and/or starch that retains a pre-defined shape for a period of time exceeding two months at room temperature. The formulation may have a substantially open pore structure that facilitates re-hydration with fluids, such as blood, autograft, allograft, bone marrow aspirate and/or mixtures and/or derivatives thereof.

In yet another exemplary embodiment, the invention provides an implant fabricated from a DBM material, gelatin, starch, glycerol and one or more porogens. Porogen particles may be made of any biocompatible, biodegradable substance that can be formed into a particle and that is capable of at least substantially retaining its shape during the manufacturing of the implant, but that is subject to rapid degradation or dissolution when placed in contact with an aqueous solution, such as an in vivo environment. The porogens may be inorganic or organic, for example, they may be made from an organic polymer (e.g., polyvinyl alcohol), a saccharide, a calcium salt, sodium chloride, calcium phosphate or mixtures thereof. Porogen particles may be about 100 to about 500 microns. In one embodiment, all porogen particles of a given morphology can have at least one average axial, transverse, or lateral dimension that is about 100 to about 500 microns. In some embodiments, all porogen particles used can independently have at least one axial, transverse, or lateral dimension that is about 100 to about 500 microns. In some embodiments, all porogen particles used can collectively have at least one average axial, transverse, or lateral dimension that is about 100 to about 500 microns.

In some embodiments, at least one dimension of the porogen particles can be about 100 microns or more, or about 120 microns or more, or about 140 microns or more. In some embodiments, at least one dimension of the porogen particles can be about 500 microns or less, about 425 microns or less, about 350 microns or less, about 300 microns or less, or about 250 microns or less. In some embodiments, the porogen particles can have at least one dimension that is about 120 to about 400 microns.

Additional exemplary embodiments, as well as features and advantages thereof, will be apparent to those skilled in the art from the descriptions herein.

### BRIEF DESCRIPTION OF THE FIGURES

FIG. 1

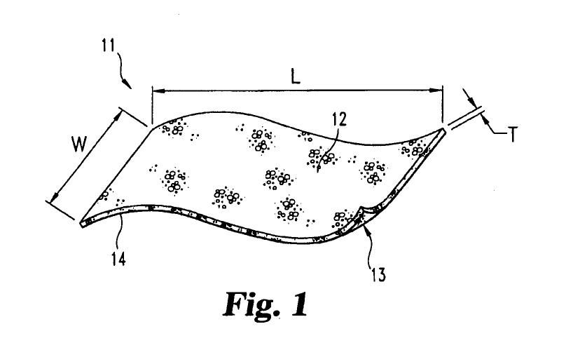

### DETAILED DESCRIPTION OF THE INVENTION

For the purposes of promoting an understanding of the principles of the invention, reference will now be made to certain embodiments and specific language will be used to describe the same. It will nevertheless be understood that no limitation of the scope of the invention is thereby intended, such alterations and further modifications in the illustrated product, and such further applications of the principles of the invention as described herein being contemplated as would normally occur to one skilled in the art to which the invention relates.

The uses of the terms “a” and “an” and “the” and similar references in the context of describing the invention (especially in the context of the following claims) are to be construed to cover both the singular and the plural, unless otherwise indicated herein or clearly contradicted by context.

As used herein, “biocompatible” means materials used elicits little or no immune response in vivo.

Recitation of ranges of values herein are merely intended to serve as a shorthand method of referring individually to each separate value falling within the range, unless otherwise indicated herein, and each separate value is incorporated into the specification as if it were individually recited herein.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “comprising,” “including,” “containing,” “characterized by,” and grammatical equivalents thereof are inclusive or open-ended terms that do not exclude additional, unrecited elements or method steps, but will also be understood to include the more restrictive terms “consisting of” and “consisting essentially of.”

The “Bloom” value of gelatin may be measured as the force in grams required to depress a standard plunger on a texture analyzer 4 mm into the surface of a 6.67% gelatin sample at 10° C. (50° F.).

Glycerol should have a min specific density of 1.26 g/ml.

The weight of compounds used herein is provide in the absence of water, hence a composition comprising about 41% DBM, about 13% starch, about 16% glycerol and about 29% gelatin would also contain a sufficient amount of an aqueous solution, such as water, to allow the compounds to be homogeneously mixed but the weight percents are expressed without regard to the water content, which may be varied both by adding water and by evaporating water.

As used herein, “about” means approximately or fairly close to.

The process of setting involves the transformation of a solution to a gel. The addition of heat to the gel to a point above the melting point can then be used to melt the gel so that a solution is formed or reformed.

For the purposes of promoting an understanding of the principles of the invention, reference will now be made to certain embodiments and specific language will be used to describe the same. It will nevertheless be understood that no limitation of the scope of the invention is thereby intended, such alterations and further modifications in the illustrated device, and such further applications of the principles of the invention as described herein being contemplated as would normally occur to one skilled in the art to which the invention relates.

As disclosed above, in certain aspects, the present invention relates to implantable medical products, and to methods for making and using the products. In particular embodiments, the osteoinductive medical implants include a three-dimensionally stable structure incorporating demineralized bone matrix (DBM) particles and at least one binding agent, such as starch and/or gelatin.

The term “demineralized bone matrix” refers to a matrix material prepared by demineralizing any bone source, including cortical and/or cancellous bone. Demineralized bone matrix materials may contain less than about 5% by weight of residual calcium, less than about 0.5% by weight of residual calcium or less than about 0.1% by weight of residual calcium. The source bone may be from any suitable source including autogenic, allogeneic, and/or xenogenic bone. When used in describing a demineralized bone matrix (DBM) material, the term “osteoinductive” refers to the ability of the DBM material to induce bone growth.

DBM materials may be obtained commercially or may be prepared by known techniques. In general, osteoinductive DBM materials may be prepared by decalcification of cortical and/or cancellous bone, often by acid extraction. This process may be conducted so as to leave collagen, noncollagenous proteins, and growth factors together in a solid matrix. Methods for preparing such bioactive DBM are well known, in respect of which reference can be made to U.S. Pat. Nos. 5,073,373; 5,484,601; and 5,284,655, as examples. DBM products are also available commercially, including for instance, from sources such as Regeneration Technologies, Inc. (Alachua, Fla.), The American Red Cross (Arlington, Va.), and others. In certain embodiments, the particulate DBM material may have an average particle size of less than about 1,000 μm. For instance, the DBM material may have particle sizes in the range of 50 to 850 μm. DBM materials may be derived from human donor tissue, especially in regard to implant devices intended for use in human subjects.

Collagen may be collagen fibers which may optionally be populated with non-native cross-links, e.g. by chemical, dehydrothermal, radiation or other cross-linking techniques. Water soluble or insoluble collagen may be used, and may be derived from natural tissue sources (e.g. xenogenic, allogenic, or autogenic relative to the recipient human or other patient) or recombinantly prepared. See U.S. Patent Pub. 20070254041, incorporated by reference.

Starch may be obtained commercially and may be derived from sources including, but not limited to, cereal grain seeds (e.g., corn, wheat, rice, and sorghum), roots and tubers (e.g., potato), legumes and other sources. In an exemplary embodiment, the starch has a lower level of endotoxin than is typically found in commercially available starches.

In an exemplary embodiment, the compositions of the invention include an amount of binding agents, such as gelatin and starch, wherein the total amount of the binding agents are less than that of the DBM on a weight-to-weight basis.

A suitable binding agent is gelatin which may have a bloom value of from about 50 to about 300 or from about 200 to about 300. Furthermore, the gelatin may be either a Type A or a Type B gelatin derived from recombinant animal or human source. Type A gelatin is obtained from acid processing of collagen and Type B gelatin is obtained from alkaline processing of collagen.

Heating may be carried out at any temperature desired and for any period of time desired, so long as the at least one binding agent, for example, gelatin, and water enter into a homogeneous solution, the temperature range to be utilized may be from about 50° C. to about 180° C. Additional agents, including additional binding agents, may be added to a heated gelatin solution, however, addition of the DBM particles is preferably conducted when the solution to which they are being added has reached a temperature below about 40° C., below about 39° C., below about 38° C., below about 37° C., below about 36° C. or below about 35° C. The heating step may be conducted for any amount of time depending upon the temperature employed in the process, the concentration of additives (including one or more binding agents) and other factors.

A moisture content of at least 10%, at least 20%, at least 30%, at least 40%, at least 50%, at least 60%, or at least 70%, total weight percent is desirable in order to facilitate formation of a homogeneous solution of the at least one binding agents, for example, an admixture of gelatin and starch or an admixture of gelatin, starch and DBM.

In certain exemplary embodiments, the sheet-form DBM composition further comprises a cosolvent, such as glycerol or polyethylene glycol.

In certain compositional embodiments of the invention, the product contains gelatin, a starch, glycerol and DBM in a liquid medium, with the gelatin present in an amount between about 17% to about 30%, between about 15% to about 32%, between about 13% to about 34% or between about 10% to about 40% (based on dry weight), the DBM is present in an amount between about 41% to about 56%, between about 40% to about 60%, between about 35% to about 65% or between about 30% to about 70% (based on dry weight), the starch is present in an amount between about 12% to about 13%, between about 11% to about 14%, between about 10% to about 15% or between about 5% to about 20% (based on dry weight) and glycerol is present in an amount between about 15% to about 17%, between about 14% to about 18%, between about 13% to about 19% or between about 10% to about 20% (based on calculated weight). The composition may also be dehydrated.

In regard to the incorporated materials considered on a dry weight basis, the gelatin may constitute about 10% to about 40% of the products. Similarly, on a dry weight basis, the compositions of the invention may contain DBM material in an amount of about 35% to about 65%. Likewise, starch may be present in an amount between about 5% to about 20%. Finally, glycerol may be present in an amount between about 10% to about 20% based on the volume added multiplied by the specific gravity. In an exemplary embodiment, the composition comprises a homogeneous admixture of gelatin in an amount from about 10% to about 40%, DBM material in an amount of about 30% to about 70%, about 35% to about 65% or about 40% to about 60%, starch in an amount between about 10% to about 15%, and glycerol in an amount between about 10% to about 20% as described herein. In another exemplary embodiment, the composition comprises a homogeneous admixture of gelatin in an amount from about 17% to about 30%, DBM material in an amount of about 41% to about 56% or about 40% to about 60%, starch in an amount between about 12% to about 13%, and glycerol in an amount between about 15% to about 7% as described herein.

In certain aspects of the invention, medical implants are provided which comprise one or more polysaccharides or starches that are non-cross-linked, non-chemically cross-linked or are cross-linked using only heat.

The starch will typically be incorporated into the implants on a dry weight basis, at total levels of about 5% to about 20%, about 10% to about 15%, or about 12% to about 13%. In certain exemplary embodiments, the DBM material may be held together to form a three-dimensional implant without the use of any chemical cross-linker, such as glutaraldehyde, formaldehyde or a carbodiimide, to covalently link the starchs and/or gelatin molecules together.

Devices of the present invention may be manufactured in a ready-to-use condition and packaged in medically acceptable packaging in sterile condition, in either wet or dry formats. In some embodiments, the ready-to-use medical product may be a porous, sheet-form product 11, resembling a resilient sponge material. Sheet-form material configured as strips or other parallelepiped three dimensional bodies are readily manufactured, and provide beneficial products. Sheet-form product 11 includes a first face 12, a second face 13, and sidewalls 14 interconnecting the first and second faces 12 and 13. Sheet-form product may have any suitable length (L), width (W) and thickness (T). In certain embodiments, the length (L) and width (W) will range from about 1 cm to about 50 cm, and the thickness T will range from about 0.1 cm to about 10 cm. More typically, the length and width will each range from about 1 cm to about 20 cm, and the thickness will be from about 0.2 cm to about 1 cm. From the standpoint of volume, preferred devices will have a body defining a volume of about 0.1 cm³ to 500 cm³, about 1 cm³ to about 100 cm³, or about 1 cm³ to about 20 cm³. It will be understood however that other linear and volumetric dimensions may also be employed within the scope of the present invention.

The bulk densities of implants made according to the present disclosure may vary depending upon factors such as the extent of their porosity, the densities of the incorporated materials, and the state of the device (e.g. hydrated or dehydrated condition). In certain embodiments, the device will be in a dried or semi-dried condition. In certain embodiments, the devices will have a bulk density in the range of about 0.1 g/cc to about 0.7 g/cc, about 0.1 g/cc to about 0.6 g/cc, about 0.1 g/cc to about 0.5 g/cc, about 0.1 g/cc to about 0.4 g/cc, about 0.1 g/cc to about 0.3 g/cc, or about 0.15 g/cc to about 0.25 g/cc. Nonetheless, as noted above these densities may vary with many factors, and bulk densities within these ranges, or that are lower or higher, may be exhibited by devices within aspects of the invention.

Some exemplary embodiments provide devices that may be substantially porous. Devices may have a void volume of at least about 40% and typically in the range of about 40% to 90%. Moderate void volume levels of about 50% to less than 70%, or higher levels in the range of 70% to 90% or more, may be controllably achieved, for instance by varying the amount of water, other liquids or cosolvents and or porogens included in the formulation used to prepare the devices.

Devices may be manufactured by preparing a mixture including the DBM material, the starch, the gelatin, along with any other liquid or solid materials to be incorporated in to the device upon original manufacture, such as glycerol. This mixture may then be placed in a mold, allowed to set, sterilized and packaged in a dried, semi-dried or wet state. Such drying may utilize lyophilization or other sublimation drying techniques. In certain embodiments, the non-chemically cross-linked admixture of materials is charged to a mold having a desired shape and allowed to set. Setting of the product might require cooling/freezing of the product for a period of time. The resultant shaped gel may be additionally processed to provide the device that is then packaged and sterilized as the final product.

The inventive devices may be implanted in the patient in a dried, semi-dried or wetted condition. External wetting agents may be applied to the devices for use in a patient, for example, aqueous substances such as sterile water, physiological saline, phosphate buffered saline, blood, bone marrow, bone marrow fractions or other liquid mediums, emulsions or suspensions that provide adequate wetting characteristics. Biocompatible organic liquids may also be used, alone or in combination with water. In desired forms, molecules of the wetting agent (e.g. water) will be taken up into the matrix to form a wetted, yet firm gel device incorporating the particulate DBM material.

Implant devices of the invention may also contain other beneficial substances including for example preservatives, cosolvents, suspending agents, buffering agents (e.g. carrying active agents to be added to the device) viscosity enhancing agents, ionic strength and osmolality adjusters and/or other excipients.

The implant devices disclosed herein may also include other biocompatible and preferably bioresorbable substances. These materials may include, for example, natural polymers such as proteins and polypeptides, glycosaminoglycans, proteoglycans, elastin, hyaluronic acid, dermatan sulfate, Alginate, or mixtures or composites thereof.

In another aspect of the invention, the devices may incorporate DBM particles having two distinct sizes, for example particles between about 1-4 mm in combination with smaller particles, for example, between about 210-710 μm.

Both human and non-human sources of bone are suitable for use in the instant invention, and the bone may be autographic, allographic or xenographic in relation to the mammal that is to receive the implant. Appropriate pre-treatments known in the art may be used to minimize the risks of disease transmission and/or immunogenic reaction when using such bone derived DBM particles.

Bioactive agents may be delivered with devices of the invention. These bioactive agents may include, for example, antimicrobials, antibiotics, antimyobacterial, antifungals, antivirals, antineoplastic agents, antitumor agents, agents affecting the immune response, blood calcium regulators, agents useful in glucose regulation, anticoagulants, antithrombotics, antihyperlipidemic agents, cardiac drugs, thyromimetic and antithyroid drugs, adrenergics, antihypertensive agents, cholnergics, anticholinergics, antispasmodics, antiulcer agents, skeletal and smooth muscle relaxants, prostaglandins, general inhibitors of the allergic response, antihistamines, local anesthetics, analgesics, narcotic antagonists, antitussives, sedative-hypnotic agents, anticonvulsants, antipsychotics, anti-anxiety agents, antidepressant agents, anorexigenlcs, non-steroidal anti-inflammatory agents, steroidal anti-inflammatory agents, antioxidants, vaso-active agents, bone-active agents, osteogenic factors, antiarthritics, and diagnostic agents.

Bioactive agents may also be provided by tissue materials incorporated into the devices, including for instance autologous or allogenic tissue materials, which are incorporated into the material to be implanted in the patient. Such tissue materials may include blood or blood fractions, bone marrow or bone marrow fractions, and/or other sources of cells or other beneficial tissue components derived from the patient to be treated or another suitable animal source. These substances may, for example, be added to the device just prior to implantation into the patient.

Bioactive agents such as those described herein may be incorporated homogeneously or regionally into the implant devices by simple admixture, soaking or otherwise.

In certain embodiments, a device of the invention will include one or more substances, additional to the osteoinductive DBM material that induces or generates the formation of bone. Suitable osteogenic materials may include a growth factor that is effective in inducing formation of bone. Desirably, the growth factor will be from a class of proteins known generally as bone morphogenic proteins (BMPs), and may in certain embodiments be recombinant human (rh) BMPs. These BMP proteins, which are known to have osteogenic, chondrogenic and other growth and differentiation activities, include rhBMP-2, rhBMP-3, rhBMP4 (also referred to as rhBMP-2B), rhBMP-5, rhBMP-6, rhBMP-7 (rhOP-1), rhBMP-8, rhBMP-9, rhBMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12, rhGDF-14. For example, BMP-2, BMP-3, BMP-4, BMP-5, BMP-6 and BMP-7, disclosed in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in U.S. Pat. No. 5,637,480; BMP-11, disclosed in U.S. Pat. No. 5,639,638, or BMP-12 or BMP-13, disclosed in U.S. Pat. No. 5,658,882, BMP-15, disclosed U.S. Pat. No. 5,635,372 and BMP-16, disclosed in U.S. Pat. Nos. 5,965,403 and 6,331,612. Other compositions which may also be useful include Vgr-2, and any of the growth and differentiation factors [GDFs], including those described in PCT applications WO94/15965; WO94/15949; WO95/01801; WO95/01802; WO94/21681; WO94/15966; WO95/10539; WO96/01845; WO96/02559 and others. Also useful in the present invention may be BIP, disclosed in WO94/01557; HP00269, disclosed in JP Publication number: 7-250688; and MP52, disclosed in PCT application WO93/16099. The disclosures of all of these Patents and applications are hereby incorporated herein by reference. Also useful in the present invention are heterodimers of the above and modified proteins or partial deletion products thereof. These proteins may be used individually or in mixtures of two or more.

The BMP may be recombinantly produced, or purified from a protein composition. The BMP may be homodimeric, or may be heterodimeric with other BMPs (e.g., a heterodimer composed of one monomer each of BMP-2 and BMP-6) or with other members of the TGF-beta superfamily, such as activins, inhibins and TGF-beta 1 (e.g., a heterodimer composed of one monomer each of a BMP and a related member of the TGF-beta superfamily). Examples of such heterodimeric proteins are described for example in Published PCT Patent Application WO 93/09229, the specification of which is hereby incorporated herein by reference. The amount of osteogenic protein useful herein is that amount effective to stimulate increased osteogenic activity of infiltrating progenitor cells, and will depend upon several factors including the size and nature of the defect being treated, and the device and particular protein being employed.

Other therapeutic growth factors or substances may also be used in devices of the present invention, especially those that may be used to stimulate bone formation. Such proteins are known and include, for example, platelet-derived growth factors, insulin-like growth factors, cartilage-derived morphogenic proteins, growth differentiation factors such as growth differentiation factor 5 (GDF-5), and transforming growth factors, including TGF-α and TGF-β.

The osteogenic proteins or other biologically active agents, when used in the present invention, may be provided in liquid formulations, for example buffered aqueous formulations. In certain embodiments, such liquid formulations may be received upon and/or within, or otherwise combined with a dry-form device by a health care provider just prior to implantation. In other embodiments, such liquid formulations may be included within wet materials used to prepare a dry-form or wetted device during its manufacture. One suitable rhBMP-2 formulation is available from Medtronic Sofamor Danek, Memphis, Tenn., with its INFUSE® Bone Graft product.

Osteoinductive devices of the present invention may also comprise progenitor and/or stem cells derived from embryonic or adult tissue sources and/or taken from culture. Illustratively, compositions of the invention may incorporate cells derived from blood, bone marrow, or other tissue sources from the patient to be treated (autologous cells) or from a suitable allogenic or xenogenic donor source. In certain embodiments of the invention, the device incorporates an enriched bone marrow fraction, prepared for example as described in US Patent Publication No. 2005/0130301 to McKay et al. published Jun. 16, 2005, which is hereby incorporated herein by reference in its entirety. Thus, implantable materials may incorporate a bone marrow fraction enriched in connective tissue growth components, that is prepared by centrifuging a biological sample (e.g. from the patient to be treated) to separate the sample into fractions including a fraction rich in connective tissue growth components. The fraction rich in connective tissue growth components may then be isolated from the separated sample, and incorporated into or onto a dry-form device of the invention, e.g. by using the fraction in or as a wetting medium applied to the device by a health care provider prior to implantation.

Osteoinductive devices of the present invention may also comprise suitable biostatic/biocidal agents including, but not limited to, antibiotics, povidone, sugars, mucopolysaccharides, chlorobutanol, quarternary ammonium compounds such as benzalkonium chloride, organic mercurials, parahydroxy benzoates, aromatic alcohols, halogenated phenols, sorbic acid, benzoic acid, dioxin, EDTA, BHT, BHA, TBHQ, gallate esters, NDGA, tocopherols, gum guaiac, lecithin, boric acid, citric acid, p-Hydroxy benzoic acid esters, propionates, Sulfur dioxide and sulfites, nitrates and nitrites of Potassium and Sodium, diethyl pyrocarbonate, Sodium diacetate, diphenyl, hexamethylene tetramine o-phenyl phenol, and Sodium o-phenylphenoxide, etc. When employed, biostatic/biocidal agent will typically represent from about 1 to about 25 weight percent of the bone particle containing composition, calculated prior to forming the shaped material. For example, the device may include one or more antibiotic drugs.

Osteoinductive devices of the present invention may also comprise suitable surface active agents, such as biocompatible nonionic, cationic, anionic and amphoteric surfactants and mixtures thereof. When employed, surface active agent will typically represent from about 1 to about 20 weight percent of the bone particle containing composition, calculated prior to forming the shaped material.

In still further embodiments, the present invention provides methods for treating patients that involve implanting in the patients an osteoinductive DBM device as described herein. In such uses, an osteoinductive DBM device may be implanted at a site at which bone growth is desired, e.g. to treat a bone disease, defect or location of trauma, and/or in some instances to promote artificial arthrodesis. The medical devices of the invention may be used as surgical implants at, in, on, or near bone defect sites, cartilage repair sites, or other musculoskeletal sites. In certain beneficial embodiments, the device will exhibit a conformable or flexible character that enables its introduction and shaping within voids, defects or other areas in which new tissue growth is desired, and/or in certain embodiments in which the delivery of a bioactive agent is desired. Further in this regard, the device may have compression-resistant properties sufficient to resist substantial compression when impinged by adjacent soft tissues of the body at a bony implant site, for instance at a posterolateral spinal fusion implant site.

Illustrative bone repair sites that may be treated with medical devices of the invention include, for instance, those resulting from injury, defects brought about during the course of surgery, infection, malignancy or developmental malformation. The devices may be used in a wide variety of orthopedic, periodontal, neurosurgical and oral and maxillofacial surgical procedures including, but not limited to: the repair of a simple fracture, compound fracture or non-union; as an external fixation device or internal fixation device; for joint reconstruction, arthrodesis, arthroplasty or cup arthroplasty of the hip; for femoral or humeral head replacement; for femoral head surface replacement or total joint replacement; for repair of the vertebral column, spinal fusion or internal vertebral fixation; for tumor surgery; for deficit filling; for discectomy; for laminectomy; for excision of spinal cord tumors; for an anterior cervical or thoracic operation; for the repairs of a spinal injury; for scoliosis, for lordosis or kyphosis treatment; for intermaxillary fixation of a fracture; for mentoplasty; for temporomandibular joint replacement; for alveolar ridge augmentation and reconstruction; as an inlay osteoimplant; for implant placement and revision; for sinus lift; for a cosmetic procedure; and, for the repair or replacement of the ethmoid, frontal, nasal, occipital, parietal, temporal, mandible, maxilla, zygomatic, cervical vertebra, thoracic vertebra, lumbar vertebra, sacrum, rib, sternum, clavicle, scapula, humerus, radius, ulna, carpal bones, metacarpal bones, phalanges, ilium, ischium, pubis, femur, tibia, fibula, patella, calcaneus, tarsal bones and/or metatarsal bones.

In accordance with certain aspects of the invention, the osteoinductive DBM device may be used as a bone void filler, or may be incorporated in, on or around load bearing implants such as spinal implants, hip implants (e.g. in or around implant stems and/or behind acetabular cups), knee implants (e.g. in or around stems). In inventive variants, the osteoinductive DBM devices may be incorporated in, on or around a load-bearing spinal implant device having a compressive strength of at least about 10000 N, such as a fusion cage, dowel, or other device potentially having a pocket, chamber or other cavity for containing the osteoinductive DBM device, and used in a spinal fusion such as an interbody fusion. One illustration of such use is in conjunction with a load-bearing interbody spinal spacer to achieve interbody fusion. In these applications, the device may be placed in and/or around the spacer to facilitate the fusion.

Illustrative cartilage repair sites that may be treated with devices of the invention include, but are not limited to, articular cartilage surfaces occurring in articular joints having at least two major bones. Examples include, but are not limited to the elbow, wrist, phalanx, knee, and ankle. Additionally, cartilage surfaces within shoulder and hip joints may be treated.

The present invention also provides medical kits and/or other products that include one or more osteoinductive DBM devices of the invention. Such products may include the device(s) of the invention, received in sterile condition in medical packaging. Such products may also include one or more additional surgical instruments or implants, for example a load-bearing implant (e.g. a spinal spacer), and/or a fluid transfer device such as a syringe, and/or a therapeutic substance, for example an osteogenic substance such as a BMP. In one specific form, such a medical kit may include a dried device of the invention, a BMP in lyophilized form (e.g. rhBMP-2), and an aqueous medium for reconstitution of the BMP to prepare an aqueous formulation that may then be added to the device.

In an exemplary embodiment, the present invention provides a method of forming an osteogenic osteoimplant, the method comprising: providing a coherent mass of DBM particles in combination with one or more biocompatible components and/or multifunctional polymers. The DBM particles may initially be in the form of a powder, fibers, chips, or a combination thereof. The method may further comprise applying heat to the composition before or during the formation of the final product. The method may further comprise forming the final product as a flexible sheet by cooling or freezing the product. The method may also comprise shaping the final product using press cutter to achieve a desired shape.

The device may comprise at least about 40% by weight of DBM and may include bone chips possessing a median size of 1 mm-4 mm, wherein the bone chips may be non-demineralized, partially demineralized, or demineralized so as to contain less than about 0.5% residual calcium.

The device may comprise a matrix of gelatin and starch, wherein any cross-linking of the matrix is limited to that occurring as a result of heating a solution containing the gelatin and/or starch or contained in the raw materials, and demineralized bone matrix particles, wherein the DBM particles are dispersed within the matrix.

Suitable cosolvents include liquid polyhydroxy compounds such as glycerol, monoacetin, diacetin, polyoxymers, block copolymers, oils, dextran, polyethylene glycol. In other exemplary embodiments the sheet-form DBM devices may include thixotropic agents such as aluminum hydroxide gel and aluminum phosphate gel, a solution of polyvinyl alcohol, polyvinylpyrrolidone, cellulosic ester such as hydroxypropyl methylcellulose, carboxyl methylcellulose, hydrolyzed polyacrylonitrile, hydrolyzed polyacrylamide, polyelectrolyte such as polyacrylic acid salt, hydrogels, gels of colloidal clays and/or mixtures thereof.

At least one binding agent will typically represent from about 30 to about 45 weight percent of the final composition.

The invention will now be more particularly described with reference to the following specific examples. It will be understood that these examples are illustrative and not limiting of the embodiments of the invention.

### Example 1

| Reagents | Quantity | DI water | Weight % |
|---|---|---|---|
| Gelatin | 2.0 g | 3 ml | 23.0% |
| Starch | 1.1 g | 7 ml | 12.6% |
| Glycerol | 1.1 g | — | 12.6% |
| DBM | 4.5 g | 9.0 ml | 51.7% |

Sample Preparation Method:

2.0 g of gelatin was measured and hydrated in 3.0 ml of DI water. In a separate beaker, the required amount of starch (1.1 g) was measured out and mixed with DI water (7 ml). To this, the required amount of glycerol (1.1 g) was added. The beaker was then placed on a heat stirrer and the solution was allowed to heat/boil while stiffing continuously. The hydrated gelatin was then added and mixed until the sample was fully dissolved. It was then allowed to boil while stiffing continuously. After boiling for a few minutes the solution was removed from the heat stirrer and allowed to cool down to about 38° C., while maintaining the sample in solution. In a separate beaker, the DBM was measured out and the required amount of DI water was added and left to allow the DBM to fully hydrate (at least 1 min). The hydrated DBM was then added to the solution maintained at 38° C. and mixed until homogenous. The mixture was then immediately poured into appropriately sized molds and allowed to set. Setting required cooling/freezing of the product for a short while. The resulting devices may then be removed from the mold, sterilized and packaged.

### Example 2

### Alternative Formulations

| Reagents | Quantity | DI water | Dry Weight % |
|---|---|---|---|
| Gelatin | 0.5 g | 3.25 ml | 28.5% |
| DBM | 1.25 g | | 71.4% |

### Example 3

### Formulation Testing

Formulations are tested using athymic male rats. Samples are randomized so that no animal receives the same lot in both implant sites. Contralateral controls are used in each rat which receive two implants; one per hind leg. The animals are anesthetized and prepared for surgery with pockets created in or between the muscle(s). The pockets are then filled with about 0.2 cc of the test article/sample and then the muscle pocket and skin are sutured closed. The animals are maintained in-life for 28 days.

At the end of the study duration, the animals are sacrificed and the implant site removed. Each implant is fixed, processed, and evaluated for histopathological evidence of new bone formation. Sections are taken from at least three levels of the test article within a block. The sections are mounted on slides for histological evaluation and a report is generated with scores for individual implant sites as either positive or negative relative to bone formation.

### Example 4

### Alternative Formulations

The following formulations were prepared as described in Example 1 above.

Formulation 4A

| Reagents | Quantity | DI water | Weight % |
|---|---|---|---|
| Gelatin | 2.5 g | 4 ml | 30.5% |
| Starch | 1.1 g | 11 ml | 13.4% |
| Glycerol | 1.1 g | — | 13.4% |
| DBM | 3.5 g | 7 ml | 42.7% |

Formulation 4B

| Reagents | Quantity | DI water | Weight % |
|---|---|---|---|
| Gelatin | 2.54 g | 6.9 ml | 30.8% |
| Starch | 1.1 g | 9.5 ml | 13.3% |
| Glycerol | 1.1 g | — | 13.3% |
| DBM | 3.5 g | 7 ml | 42.5% |

Formulation 4C

| Reagents | Quantity | DI water | Weight % |
|---|---|---|---|
| Gelatin | 2.54 g | 5 ml | 29.1% |
| Starch | 1.1 g | 11 ml | 12.6% |
| Glycerol | 1.1 g | — | 12.6% |
| DBM | 4.0 g | 8.0 ml | 45.8% |

Formulation 4D

| Reagents | Quantity | DI water | Weight % |
|---|---|---|---|
| Gelatin | 1.5 g | 2.5 ml | 18.3% |
| Starch | 1.1 g | 7 ml | 13.4% |
| Glycerol | 1.1 g | — | 13.4% |
| DBM | 4.5 g | 9 ml | 54.9% |

Formulation 4E

| Reagents | Quantity | DI water | Weight % |
|---|---|---|---|
| Gelatin | 1.75 g | 2.5 ml | 20.7% |
| Starch | 1.1 g | 7 ml | 13.0% |
| Glycerol | 1.1 g | — | 13.0% |
| DBM | 4.5 g | 9 ml | 53.3% |

Formulation 4F

| Reagents | Quantity | DI water | Weight % |
|---|---|---|---|
| Gelatin | 1.5 g | 2 ml | 17.2% |
| Starch | 1.1 g | 7 ml | 12.6% |
| Glycerol | 1.1 g | — | 12.6% |
| DBM | 5.0 g | 10 ml | 57.5% |

Formulation 4G

| Reagents | Quantity | DI water | Weight % |
|---|---|---|---|
| Gelatin | 1.75 g | 2.5 ml | 19.6% |
| Starch | 1.1 g | 7 ml | 12.3% |
| Glycerol | 1.1 g | — | 12.3% |
| DBM | 5.0 g | 10 ml | 55.9% |

### Example 5

### Formulation Testing

Formulations 4F and 4G from Example 4 were tested using athymic male rats generally as described herein. At the end of the study duration, the animals were sacrificed and the implant site removed. Each implant was processed and evaluated for evidence of new bone formation.

| | | Chondroblasts | Osteoblasts | Cartilage | | |
|---|---|---|---|---|---|---|
| | Animal | or | or | or | New | Bone |
| Formulation | location | Chondrocytes | Osteocytes | Osteoid | Bone | Marrow |
| 4G | LL | X | X | X | X | X |
| 4G | LL | X | X | X | X | X |
| 4G | LL | X | X | X | X | X |
| 4G | LL | X | X | X | X | X |
| 4F | LL | X | X | X | X | X |
| 4F | RL | X | X | X | X | X |
| 4F | RL | X | X | X | X | X |

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all publications cited herein are hereby incorporated by reference in their entirety.

What is claimed is:

1. A sheet-form demineralized bone matrix (DBM) composition, comprising:
an aqueous admixture of DBM particles, starch and gelatin;
wherein the DBM particles are present in an amount of between about 30% and about 70% by weight;
wherein the gelatin is present in an amount between about 10 and about 40% by weight;
wherein the starch is present in an amount between about 5% and about 20% by weight; and
wherein the DBM particles are retained in the form of the resilient sheet.

2. The sheet-form DBM device of claim 1, wherein said resilient sheet is in a semi-dried state.

3. The sheet-form DBM device of claim 1, wherein the gelatin is present in an amount between about 17% to about 30% by weight.

4. The sheet-form DBM device of claim 3, wherein the starch is present in an amount between about 11% and about 14% by weight.

5. The sheet-form DBM device of claim 1, further comprising a cosolvent in an amount of at least 10% by weight.

6. The sheet-form DBM device of claim 5, wherein the cosolvent is glycerol.

7. The sheet-form DBM device of claim 6, wherein the glycerol is present in an amount of between 15% and 17% by weight.

8. The sheet-form DBM device of claim 7, wherein the DBM comprises between about 40% to about 60% by weight.

9. The sheet-form DBM device of claim 1, wherein the DBM comprises between about 40% to about 60% by weight.

10. The sheet-form DBM device of claim 1, further comprising bone chips greater than 1 mm in size.

11. The sheet-form DBM device of claim 10, wherein the bone chips are partially demineralized.

12. The sheet-form DBM device of claim 1, wherein the DBM particles are obtained from cortical, cancellous or cortico-cancellous types of bone, said types of bone being of autogenous, allogenic or xenogeneic origin.

13. A method for preparing a demineralized bone matrix (DBM) implant, comprising:
preparing an aqueous admixture of gelatin and starch;
heating the aqueous admixture;
stirring the aqueous admixture;
cooling the aqueous admixture to a temperature below about 40° C.;
adding DBM particles to the aqueous admixture maintained below about 40° C. to produce a homogeneous DBM mixture; and
poring the homogeneous DBM mixture into a mold or form.

14. The method according to claim 13, further comprising at least partially drying the homogeneous DBM mixture.

15. The method according to claim 13, wherein preparing an aqueous admixture of gelatin and starch comprises preparing an aqueous admixture containing gelatin, starch and a cosolvent.

16. The method according to claim 13, wherein preparing an aqueous admixture of gelatin and starch comprises preparing an aqueous admixture containing gelatin, starch and a cosolvent.

17. The method according to claim 17, wherein preparing an aqueous admixture of gelatin and starch comprises preparing an aqueous admixture having between about 10% to about 15% starch by weight and between about 10% and about 40% of gelatin by weight.

18. The method according to claim 17, further comprising adding glycerol in an amount of between about 10% to about 20% by weight.

19. The method according to claim 17, wherein the DBM comprises between about 30% to about 70% by weight.

20. The method according to claim 13, further comprising adding bone chips greater than 1 mm in size.
